(19) United States
(12) Patent Application Publication
(10) Pub. No.: US 2011/0244016 A1
(43) Pub. Date: Oct. 6, 2011

(54) MEDICAL IMPLANT HAVING A COATING COMPOSED OF OR CONTAINING AT LEAST ONE ACTIVE SUBSTANCE

(76) Inventors: Matthias Gratz, Erlangen (DE); Alexander Borck, Aurachtal (DE); Kathleen Koeck, Putbus (DE)

(21) Appl. No.: 13/070,805

(22) Filed: Mar. 24, 2011

Related U.S. Application Data
(60) Provisional application No. 61/318,806, filed on Mar. 30, 2010.

(51) Int. Cl. A61F 2/82; A61K 31/40; A61K 9/00; C07D 207/34; A61P 9/14

(52) U.S. Cl. 424/425; 514/423; 424/423; 424/426; 548/537

(57) ABSTRACT

One embodiment of the invention relates to a medical implant whose surface is completely or partially covered by a coating composed of at least one active substance or containing at least one active substance.

### RELATED APPLICATION

This patent application claims the benefit of U.S. Provisional Patent Application No. 61/318,806, filed on Mar. 30, 2010, which is hereby incorporated by reference in its entirety.

### TECHNICAL FIELD

The invention relates to a medical implant whose surface is completely or partially covered by a coating composed of at least one active substance or containing at least one active substance.

### BACKGROUND

Implants have found use in modern medical technology in many different embodiments. They are used, for example, for supporting vessels, hollow organs, and duct systems (endovascular implants), for attaching and temporarily fixing tissue implants and tissue transplants, as well as for orthopedic purposes, for example as pins, plates, or screws, as well as for many other applications.

The implant has a base body made of an implant material. An implant material is a nonliving material which is used for medical applications and interacts with biological systems. A requirement for use of a material as an implant material, which when properly used is in contact with the bodily surroundings, is compatibility with the body (biocompatibility). Biocompatibility is understood to mean the ability of a material to induce an appropriate tissue reaction in a specific application. This includes adaptation of the chemical, physical, biological, and morphological surface characteristics of an implant to the recipient tissue, with the objective of a clinically sought interaction. The biocompatibility of the implant material is also dependent on the time sequence of the reaction of the biosystem which has received the implant. Relatively short-term irritation and inflammation occur which may result in changes in the tissue. Accordingly, biological systems respond in various ways, depending on the characteristics of the implant material. Implant materials may be roughly divided into bioactive, bioinert, and degradable/absorbable materials, depending on the reaction of the biosystem.

A biological reaction to polymeric, ceramic, or metallic implant materials depends on the concentration, duration of effect, and type of delivery. The presence of an implant material frequently results in a biological reaction, which may be inflammatory in nature, and which may be initiated by mechanical irritants, chemical substances, or metabolites. The inflammation process is generally accompanied by migration of neutrophilic granulocytes and monocytes through the vessel walls, migration of lymphocyte effector cells with formation of specific antibodies against the inflammatory irritant, activation of the complement system with release of complement factors which act as mediators, and lastly, activation of blood dotting. An immunological response is usually closely linked to the inflammation reaction, and may result in sensitization and development of allergies. Known metallic allergens include nickel, chromium, and cobalt, for example, which are also used in many surgical implants as alloy components. A significant problem with stent implantation in blood vessels is in-stent restenosis. The intravascular intervention may lead to increased thrombus formation and increased proliferation of smooth muscle cells, which may result in renewed vascular occlusion (restenosis). After extended periods, excessive proliferation of scar tissue results in restenosis in approximately 30-40% of all uncoated stents.

Biocorrodible metals and their alloys have been proposed for use as implant material For example, the production of medical implants from a metallic material whose primary component is iron, zinc, or aluminum, or an element from the group of alkali metals or alkaline earth metals has been proposed. Alloys based on magnesium, iron, and zinc have also been proposed. Secondary components of the alloys may be manganese, cobalt, nickel, chromium, copper, cadmium, lead, tin, thorium, zirconium, silver, gold, palladium, platinum, silicon, calcium, lithium, aluminum, zinc, and iron. Use of a biocorrodible magnesium alloy containing >90% magnesium, 3.7-5.5% yttrium, 1.5-4.4% rare earth metals, and the remainder <1%, has also been proposed for producing an endoprosthesis, for example in the form of a self-expanding or balloon-expandable stent.

However, many of these proposed biocorrodible implant materials have reduced mechanical integrity due to the high mechanical stress and/or vasospasms caused, among other factors, by the degradation products of the active substance, which results in the implants losing their support capability earlier than necessary.

Implants and stents may also have a coating or cavity filling with a suitable polymer. Another possibility for preventing the risk factors of restenosis is the development of a variety of coatings for stents which are designed to provide increased hemocompatibility. Vasodilative, anticoagulant, antimicrobial, anti-inflammatory, and antiproliferative agents as well as active substances which, individually or in combination, avoid or even prevent the risk of restenosis have been proposed including in the coating for stents, for example.

However, many coated stents have the disadvantage that, on account of their limited biological properties, for successful treatment at the implant tissue the particular active substances must either be used in a higher concentration, which may result in local intoxication, or applied to or introduced into the coating in combination with other active substances, resulting in increased production effort and expense.

Various active substances are known to have inflammation-inhibiting, anti-thrombotic, and anti-platelet activity, and may also be used for reducing cholesterol and triglyceride levels, raising the HDL-C levels, and treatment and prevention of acute coronary syndromes, stroke, peripheral arteriosclerotic vascular diseases, and all disorders associated with endothelial dysfunction, for example vascular conditions in diabetic patients and arteriosclerosis. These substances also have suitable properties for treatment of neurodegenerative and autoimmune diseases, for example Alzheimer's disease and Parkinson's disease, as well as multiple sclerosis.

However, systemic administration of active substances has the disadvantage that the active substances are not delivered in a targeted manner to the tissue to be treated, so that long-term, multiple administrations of active substance are generally necessary before, during, and/or after the implantation.

### SUMMARY

One embodiment of the invention is a medical implant, wherein the surface of the implant is at least partially covered by a coating containing one or more active substances, wherein at least one of the active substances is a nitrostatin.

### DETAILED DESCRIPTION

Before discussing example embodiments of the invention in detail, it will be appreciated that the invention includes not only implants but also methods for making implants. It will be appreciated that in discussing example stents of the invention, description may also be had of example methods for making those stents, and vice versa.

A feature of some embodiments of the present invention is to reduce or eliminate one or more of the described disadvantages of the prior art.

This feature is achieved according to some embodiments of the invention by completely or partially covering a medical implant with a coating composed of at least one active substance or containing at least one active substance, wherein the active substance is a nitrostatin.

One advantage of the approach according to the invention is that nitrostatins have a targeted antithrombotic, anti-inflammatory, and antiproliferative effect at the implantation site.

It was a surprising discovery that, because of the release of NO into the surrounding implant tissue, nitrostatins also have an excellent vasodilative effect, so that vasoconstriction is counteracted directly at the site due to the release of active substance from the coating of the implant according to the invention. This characteristic of the implants according to the invention is particularly advantageous when the implant is made of a biocorrodible material, since longer service lives of the implants may thus be achieved, and when the implant according to the invention is a stent, for example, the risk of restenosis or damage of the vessel lumen in and around the stent region may be greatly reduced. Thus, by use of the implant the therapeutic aim being pursued may be achieved more satisfactorily, over a longer period of time, and with fewer side effects.

Medical implants within the scope of protection of the present invention encompass any given medical devices which are used, at least in part, for introduction into the body of a patient. Examples of implantable devices include (but are not limited to) cardiac pacemakers, catheters, needle injection catheters, blood clot filters, vascular transplants, balloons, stent transplants, bile duct stents, intestinal stents, bronchial lung stents, esophageal stents, ureteral stents, aneurysm filling coils and other coil devices, transmyocardial revascularization devices, and percutaneous myocardial revascularization devices. In addition, any given natural and/or synthetic medical products may be used, for example (but not by way of limitation) prostheses, organs, vessels, aortas, cardiac valves, tubes, organ replacement parts, implants, fibers, hollow fibers, membranes, preserved blood, blood containers, titer plates, adsorbent media, dialyzers, connecting pieces, sensors, valves, endoscopes, filters, pump chambers, and other medical products which are intended to have hemocompatible properties. The term “medical products” is to be broadly construed, and refers in particular to products which come into contact with blood on a short-term basis (endoscopes, for example) or a long-term basis (stents, for example), and also a combination of individual medical implants, for example a stent and a balloon catheter, as well as implantable defibrillators, cardiac pacemakers, pacemaker electrodes, and diagnostic catheters.

Balloon catheters and stents may find particularly beneficial advantages according to some embodiments of the invention.

Stents of conventional design have a filigreed support structure made of metallic struts, which initially are in an unexpanded state for insertion into the body, and are then widened into an expanded state at the site of application. The stent may be coated before or after crimping onto a balloon.

The base body of the stent may be made of a metallic material composed of one or more metals from the group comprising iron, magnesium, nickel, tungsten, titanium, zirconium, niobium, tantalum, zinc, or silicon, and optionally a second component composed of one or more metals from the group comprising lithium, sodium, potassium, calcium, manganese, iron, or tungsten, and further may be composed of a zinc-calcium alloy.

In a further embodiment, the base body is made of a shape memory material composed of one or more materials from the group comprising nickel-titanium alloys and copper-zinc-aluminum alloys, and may be further composed of nitinol.

In further embodiments the base body of the stent may be made of stainless steel, including, but not limited to a Cr—Ni—Fe steel, the alloy 316L, or a Co—Cr steel. The base body of the stent may also be composed, at least partially, of plastic and/or a ceramic.

In a further embodiment, the base body of the stent is composed of a biocorrodible metallic active substance, for example a biocorrodible alloy, selected from the group comprising magnesium, iron, and tungsten; and may further include a biocorrodible magnesium alloy.

A biocorrodible magnesium alloy is understood to mean a metallic structure having magnesium as the primary component. The primary component is the alloy component having the highest proportion by weight in the alloy. A proportion of the primary component may be greater than 50% by weight, and may further be greater than 70% by weight, or may be other percentages by weight. The biocorrodible magnesium alloy may contain yttrium and other rare earth metals, since such an alloy is well-suited due to its physicochemical properties and high biocompatibility, in particular also its degradation products. Further, a magnesium alloy may be used having a composition of 5.2-9.9% by weight of rare earth metals, of which yttrium constitutes 3.7-5.5% by weight, and the remainder <1% by weight, wherein magnesium makes up the remaining proportion of the alloy to give 100%. This magnesium alloy has been experimentally proven, and its particular suitability, i.e., high biocompatibility, favorable processing characteristics, good mechanical parameters, and corrosion characteristics, has been demonstrated in initial clinical trials. In the present context, the collective term “rare earth metals” refers to scandium (21), yttrium (39), lanthanum (57), and the following 14 elements following lanthanum (57): cerium (58), praseodymium (59), neodymium (60), promethium (61), samarium (62), europium (63), gadolinium (64), terbium (65), dysprosium (66), holmium (67), erbium (68), thulium (69), ytterbium (70), and lutetium (71).

In a further embodiment, the stent is composed of natural polymers, for example collagen, chitin, chitosan, and heparin.

In one embodiment, a balloon catheter may be used. Balloon catheters are used in many fields of medical technology. The use of balloon catheters is a preferred therapeutic method for various indications. In angiology and cardiology, for example, balloon dilation is recommended for expansion of constricted blood vessels.

The basic design of balloon catheters is known to one having ordinary skill in the art, and are described, for example, in U.S. Pat. No. 5,522,882 A and U.S. Pat. No. 7,217,278 B2, which are hereby incorporated by reference. One form of a balloon catheter used for expanding abnormally constricted vessels in the body or for placing vessel wall supports (referred to as “stents”), have an outer shaft with a distal end and an inner shaft, situated therein to form an annular fluid line, which projects beyond the distal end of the outer shaft. At the distal end of the catheter, a balloon is attached at its proximal end in a fluid-tight manner to the distal end region of the outer shaft at a first attachment zone, and at its distal end is attached in a fluid-tight manner to the distal end region of the inner shaft at a second attachment zone. In the undilated state the balloon is placed between these attachment zones in longitudinal folds in order to minimize its outer diameter in this state. This is necessary to allow the balloon catheter together with the balloon to be pushed at the distal end through narrow vessels or sharply curved vessel regions. After the balloon is set in position at the application site, a fluid under pressure may be introduced through the annular fluid line formed between the inner and outer shafts, and the balloon may be dilated. This causes the longitudinal folds to unfold in the peripheral direction, with a large increase in the diameter of the balloon.

Some balloon catheters are made from semicrystalline thermoplastics; primarily used in the PTCA/PTA area are polyamides, polyethylene terephthalate (PET), polybutylene terephthalate (PBT), or polyether polyurethanes, and copolymers and blends thereof. Polyurethanes are being increasingly used as alternative materials for expandable and adjustable balloon applications.

Within the meaning of the invention, a coating is an application, at least in places, of the components of the coating on the base body of the implant. The entire surface of the base body of the implant may be covered by the coating, or only sleeted portions of the base body. The layer thickness may be in the range of 1 nm to 100 μm, further 300 nm to 15 μm, and still further 5 μm to 60 μm, or other thicknesses, including less than 1 nm or greater then 100 μm. Thickness in the 5 μm to 60 μm range are believed to provide superior results in at least many applications. The coating may contain a polymer which is able to form a carrier matrix. The proportion by weight of such a polymer matrix in the components forming the coating may be at least 40%, further at least 70%, or other percentages including less then 40%. The proportion by weight of the at least one nitrostatin in the components forming the coating may be less than 60%, further less than 30%, and may be in a range of 10% to 25%. Such concentration ranges are believed to provide superior results in at least some applications. Other composition percentages may also be used in some other embodiments, including but not limited to greater than 60%. The coating may be applied directly to the implant surface. Processing may be carried out according to standard methods for the coating, including but not limited to spraying, dipping, and others. Monolayer as well as multilayer systems (for example, so-called base coat, drug coat, or top coat layers) may be produced. The coating may be applied directly to the base body of the implant, or additional layers used for bonding, for example, are provided therebetween.

Alternatively, the coating composed of or containing at least one nitrostatin may be present as a cavity filling or as a component of a cavity filling. For this purpose an implant, further a stent, has one or more cavities. Cavities are located at the surface of the implant, for example, and may be produced, for example, by laser ablation in the nano- to micrometer range. For implants, further stents, having a biodegradable base body a cavity may also be provided in the interior of the base body so that the material is not released until exposure is made to a biological material only when the interior of the base body is exposed to the biological material after being arranged in a desired implanted location. One skilled in the art may refer to systems described in the prior art in designing the cavity. The term “cavity” includes holes and recesses, for example.

The embodiment of a coating as cavity filling may be of particular interest when larger quantities of the at least one nitrostatin are to be delivered to the implant tissue over an extended period of time. “Extended periods of time” as used herein may refer to periods of several hours, several days, several weeks, several months, or other periods. The active substance loading on the implant may be increased by 50% or other amounts by filling cavities.

The at least one nitrostatin may be present embedded in a polymeric carrier matrix. Within the scope of the present invention, the carrier matrix is a biostable and/or biodegradable polymer layer, the polymers being selected from the group comprising nonabsorbable permanent polymers and/or absorbable biodegradable polymers.

The carrier matrix may be composed of polymers selected from the group comprising: polyolefins, polyether ketones, polyethers, polyvinyl alcohols, polyvinyl halides, polyvinyl esters, polyvinyl pyrrolidone, polyacrylates, polyhaloolefins, polyamides, polyamidimides, polysulfones, polyesters, polyvinyl ethers, polyurethanes, silicones, polyphosphazenes, polyphenylene, polymer foams (composed of styrenes and carbonates), polydioxanones, polyglycolides, polylactides, poly-ε-caprolactone, ethyl vinyl acetate, polyethylene oxide, polyphosphorylcholine, polyhydroxybutyric acids, lipids, polysaccharides, proteins, polypeptides, and copolymers, blends, and derivatives of these compounds.

The carrier matrix may be composed of polymers selected from the group comprising: polypropylene, polyethylene, polyisobutylene, polybutylene, polyether ether ketone, polyethylene glycol, polypropylene glycol, polyvinyl alcohols, polyvinyl chloride, polyvinyl fluoride, polyvinyl acetate, poly(vinyl isobutyl ether), polyvinyl pyrrolidone, polyethyl acrylate, polymethyl acrylate, poly(methylmethacrylate), poly(ethylmethacrylate), poly(butylmethacrylate), poly(isobutylmethacrylate), poly(hexylmethacrylate), poly(phenylmethacrylate), poly(isopropylacrylate), poly(isobutylacrylate), poly(octadecylacrylate), polytetrafluoroethylene, polychlorotrifluoroethylene, PA 11, PA 12, PA 46, PA 66, polyamidimides, polyethersulfone, polyphenylsulfone, polycarbonates, polybutylene terephthalate, polyethylene terephthalate, elastanes, pellethanes, silicones, polyphosphazenes, polyphenylene, polymer foams (composed of styrenes and carbonates), polydioxanones, polyglycolides, poly-L-, poly-D-, and poly-D,L-lactide, and poly-ε-caprolactone, ethyl vinyl acetate, polyethylene oxide, polyphosphoryl choline, polyhydroxyvalerate, cholesterol, cholesterol ester, alginate, chitosan, levan, hyaluronic acid, uronides, heparin, dextran, methylcellulose, ethylcellulose, hydroxypropylcellulose, hydroxypropylmethylcellulose, hydroxybutylmethylcellulose, cellulose acetate, cellulose propionate (with low, medium, or high molecular weight), cellulose acetate propionate, cellulose acetate butyrate, cellulose acetate phthalate, carboxymethylcellulose, cellulose triacetate, cellulose sulfate sodium salt, fibrin, albumin, polypeptides, and copolymers, blends, and derivatives of these compounds. Other polymers may be used.

The carrier matrix may conform to the desired elution speed and the individual characteristics of the various active substances used, as well as the differing absorption or degradation speed at the site of action of the medical implant.

In one embodiment, the active substance is delivered from the coating of the implant surface, containing at least one nitrostatin, into the surrounding implant tissue over a period between 2 seconds and greater than 2 weeks, further between 2 seconds and greater than 6 weeks. In some embodiments, delivery occurs over a period of greater than 2 weeks, and in others in a period of greater than 6 weeks. In some embodiments, the coating is configured so that release of the nitrostatin occurs at a generally linear release rate. This can be achieved, for example, by adjusting the coating laodding, thickness and/or concentration of nitrostatin therein.

In a further embodiment, the active substance is delivered from the coating of a balloon catheter into the surrounding implant tissue over a period between 1 and 90 seconds, further between 1 and 30 seconds. In this case a pharmaceutical adjuvant may also be applied to and/or introduced into the coating.

Within the scope of the present invention a pharmaceutical adjuvant is, for example, benzalkonium chloride, α-tocopherol, glucose, lactose, calcium phosphate, calcium hydrogen phosphate, sodium hydrogen carbonate, sodium carbonate, titanium oxide, zinc oxide, magnesium oxide, silicates such as highly dispersed silica (colloidal silicic acid, Aerosil, SiO₂), talc, kaolin, bentonite; aliphatic alcohols, DMSO, glycerol, propylene glycol, stearic acid, sugar and sugar alcohols, lactose, cyclodextrins such as α-cyclodextrin, β-cyclodextrin, and γ-cyclodextrin; mannitol, sorbitol, starches, cellulose powder, cellulose esters and ethers such as methylcellulose, ethylcellulose, hydroxyethylcellulose, hydroxypropylcellulose, hydroxypropylmethylcellulose, sodium carboxymethylcellulose, cellulose acetate phthalate, hydroxypropylmethylcellulose acetate phthalate, and microcrystalline cellulose, gelatins; squalene and other isoprene units, gum arabic, pectin, xanthan, alginates, shellac, polyacrylic acids such as carbomers and Eudragit®, polyvinyl pyridine, polyvinyl pyrrolidone, polyvinyl alcohol, polyvinyl acetate, and mixtures of polyvinyl pyridine and polyvinyl acetate, polyethylene glycol, Vaseline, synthetic and natural fats and silicones, amphiphiles or surface-active adjuvants such as anion-active surfactants, and saponides; cationic surfactants such as cetylpyridinium chloride, noniogenic surfactants; polyoxyethylene sorbitan, macrogolglycerol fatty acid esters, fatty alcohol ethers of polyoxyethylene, fatty acid esters of sucrose, and in particular D-α-tocopheryl 1000 succinate, amphoteric surfactants, complex emulsifiers such as cetyl stearyl alcohol (types A and B), quaternary ammonium compounds, and preservatives and antioxidants such as citric acid, citraconic acid, tartaric acid, mono- and polyphosphates, organic phosphates such as dodecyl phosphate, hexose phosphate, and hyaluronidases or hyaluronate lyases, butyryl-n-trihexylcitrate (BTHC,) and triethyl citrate. The proportion by weight of such a pharmaceutical adjuvant in the components forming the coating may be at least 0.1%, further at least 2%, or other percentages.

Some derivatives of statin, nitrostatitns or statin nitrodeerivatives have an improved pharmacological efficacy as compared to natural statins. When systematically administered, these compounds have inflammation-inhibiting, anti-thrombotic, and anti-platelet activity, and may also be used for reducing cholesterol and triglyceride levels, raising the HDL-C levels, and treatment and prevention of acute coronary syndromes, stroke, peripheral arteriosclerotic vascular diseases, and all disorders associated with endothelial dysfunction, for example vascular conditions in diabetic patients and arteriosclerosis. These substances also have suitable properties for treatment of neurodegenerative and autoimmune diseases, for example Alzheimer's disease and Parkinson's disease, as well as multiple sclerosis. Some embodiments of the present invention exploit the advantages of these materials in unique and beneficial ways.

Within the scope of the present invention, the at least one nitrostatin includes (but is not limited to) a compound of general formula (I) and a pharmaceutically acceptable salt or stereoisomer thereof:

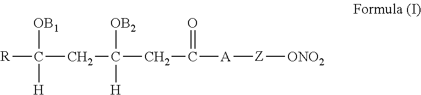

where R is the statin radical (also statin component) described below, A is as defined below, and Z is one of the suitable linking groups defined below.

R, B₁, B₂, Z, and A in general formula (I) have the following meanings according to at least some embodiments of the invention:

R is

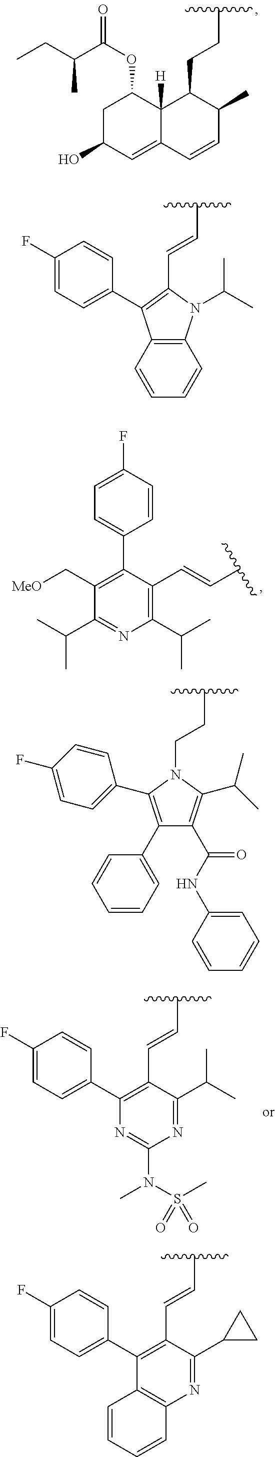

B₁ and B₂ may be chosen from:

a) a further NO-releasing group such as —NO₂, —ONO₂, or cinidomine (II),

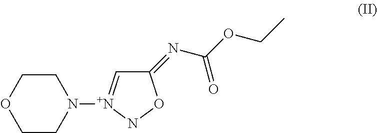

b) a tosylate or mesylate group,

c) a straight-chain or branched C₁ to C₂₀ alkyl, preferably C₁ to C₁₀ alkyl, optionally substituted with one or more substituents selected from the group comprising the following: halogen atoms, hydroxy, —ONO,

d) a heterocyclic saturated, unsaturated, or aromatic ring containing one or more heteroatoms selected from nitrogen, oxygen, sulfur, or halogen atoms, the ring being additionally substituted with side chains having a straight-chain or branched alkyl containing 1 to 10 carbon atoms,

e) cycloalkylene or cycloarylene containing 5 to 7 carbon atoms in the cycloalkylene or cycloarylene ring, the ring being substituted with side chains having a straight-chain or branched alkyl containing 1 to 10 carbon atoms, and optionally containing one or more heteroatoms selected from nitrogen, oxygen, sulfur, or halogen atoms,

wherein B₁ may be the same as or different from B₂.

A is a straight-chain or branched C₁ to C₂₀ alkylene, preferably C₁ to C₁₀ alkyl, in particular —CH₂—; —O—, —S—, —NH—, or NR¹, wherein R¹ is a straight-chain or branched C₁-C₁₀ alkyl, preferably CH₃; A is very particularly preferably —CH₂—, —O—, or —S—.

Z is a bivalent radical having the following meaning:

a) straight-chain or branched C₁-C₂₀ alkylene, preferably C₁-C₁₀ alkylene, optionally substituted with one or more substituents selected from the following group: halide, —OH, —OR¹, —COOH, —COOR¹, —NH₂, NHR¹, NR¹R², wherein R¹ and R² are the same or different, and are a straight-chain or branched C₁-C₁₀ alkyl,

b) cycloalkylene containing 5 to 7 carbon atoms in the cycloalkylene ring, wherein the cycloalkylene ring is optionally substituted with one or more straight-chain or branched C₁-C₁₀ alkyl side chains,

c) arylalkylene, in particular

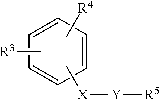

wherein R³ is (CH₂)ₙ₁, where n1=0 or 20; R⁴ is —COOH, —COOR¹, —OH, —OR¹, where R¹ is defined as above; X is a saturated or unsaturated —(CH₂)ₘ group, where m=0-8; Y═—O—CO—, —COO—; and R⁵ is (CH₂)ₙ₂, where n2=1-20,

d) Heterocycloalkylene having a saturated, unsaturated, or aromatic 5- or 6-membered ring containing one or more heteroatoms selected from nitrogen, oxygen, or sulfur, in particular the following groups:

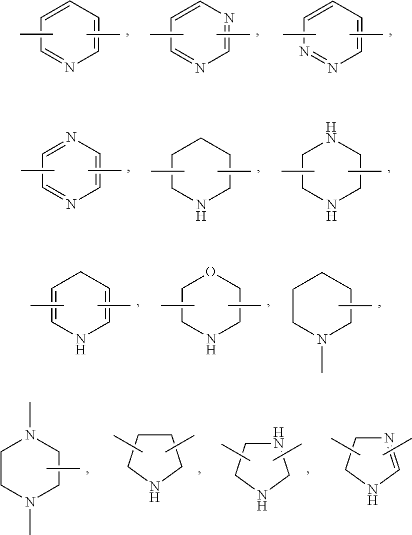

e) —O—, —Sᵢ—, —Se—, —NH—, or NR¹, where i is an integer between 1 and 8 and R¹ is a straight-chain or branched C₁-C₁₀ alkyl, preferably CH₃.

In some embodiments the statin radical R in general formula (I) may be selected in particular from the group comprising atorvastatin, rosuvastatin, pravastatin, fluvastatin, lovastatin, pitavastatin, simvastatin, and cerivastatin.

The following groups R, A, and Z may prove to be of particular utility in at least some embodiments:

### A CH₂, O, S

### Z O, S

For many nitrostatins, the nitrate esters of the following formula are responsible for the release of NO:

R—O—N⁺(O)—O⁻. This frequently occurring structural pattern is particularly suited for NO splitting.

Cinidomine has special status as an NO-releasing compound. This is a prodrug that is converted in the liver. This metabolite then splits NO without enzyme catalysis.

It has been discovered that the compound class of nitrostatins has superior properties for medical implants according to the invention and corresponding embodiments of the invention achieve unique advantages and benefits. This is at least partly due to the specific structure of these compounds, which may be subdivided into two highly potent components: a statin radical and an NO component formed during metabolism. Nitrostatins therefore have a broad pharmacological activity spectrum.

The preparation of nitrostatins is known to one skilled in the art, need not be discussed in detail herein and is therefore addressed here only briefly. Statins containing a carbonyl function may be easily converted to nitrostatins. Halides may be converted to nitrostatin in a very elegant manner in good yields, using NO₂⁺BF₄⁻. Alternatively, under less mild conditions the conversion may be carried out using silver nitrate. For obtaining halides, a Hunsdiecker reaction is suited in which the —COOH group is reacted with Ag⁺ in CCl₄, followed by quenching with Br₂, which results in the halide. Alcohols may be converted to the nitrate ester group even more easily. In this case reaction with HNO₃, possibly HNO₃ and H₂SO₄ (nitrating acid), is suitable. Due to the by-products which occur, this reaction pathway is less suitable than the conversion to alkyl halides and subsequent reaction with silver nitrate (AgNO₃) or the preferred NO₂⁺BF₄⁻. Besides the described Hunsdiecker reaction for obtaining halides, the variant of reacting the acid group with HgO in the presence of Br₂ is feasible. Chlorohalides may be obtained using the Kochi reaction. The —COOH group is reacted with lithium chloride (LiCl) in the presence of lead tetraacetate (Pb(OAc)₄). Methods of obtaining these derivatives are further described in textbooks of organic chemistry as well as many other references.

The statin component has antithrombotic, anti-inflammatory, and antiproliferative properties which are particularly useful for the treatment of irritated or slightly damaged implant tissue. Statins also inhibit cholesterol synthesis, and therefore retard the local progression of arteriosclerosis. Statins also promote the positive composition of plaque or plaque precursors as well as intensified recruitment of endothelial precursor cells from the bone marrow, which accelerates regeneration of the endothelium at the implant tissue and in turn suppresses the damage-related neointima formation. Embodiments of the present invention exploit these properties in unique and beneficial ways.

The NO component, which is released into the surrounding implant tissue, has a superior vasodilative effect, so that vasoconstriction is counteracted directly at the site due to the release of active substance from the coating of the implant according to the invention. This characteristic is particularly advantageous when the medical implant is made of a biocorrodible active substance such as magnesium or an alloy thereof. When the biocorrodible magnesium alloy degrades, its degradation products cause alkalosis at the implantation site, which may result in localized vasospasms. In addition to the mechanical stress, vasoconstriction reduces the service life of the implant, which often is not desired. Longer service lives of the implants may be achieved due to the vasodilative effect of the NO component. It is also advantageous to use a nitrostatin which contains multiple NO-releasing groups. The vasodilative effect may thus be increased and adapted to the particular implantation site which may otherwise possibly subject the implant to greater stress.

If the implant according to the invention is a stent, the risk of restenosis or damage to the vessel lumen in and around the stent region may be greatly reduced. Thus, by use of the implant the therapeutic aim being pursued may be achieved more satisfactorily, over a longer period of time, and with fewer side effects.

The at least one nitrostatin may be contained in a pharmaceutically active concentration of 0.2-3.5 μg/mm² implant surface, further, 0.5-1.6 μg/mm² implant surface, or other concentrations that may be greater or less than these.

In addition to the at least one nitrostatin, the medical implant according to the invention may include one or more further pharmaceutical active substances which are delivered to the surroundings of the implant and optionally released at very low rates into the bloodstream. The further pharmaceutical active substance may include one or more compounds selected from the following drug classes: antimicrobial, antimitotic, antimyotic, antineoplastic, antiphlogistic, antiproliferative, antithrombotic, and vasodilatory agents, or may include other compounds.

Further pharmaceutical active substances may include triclosan, cephalosporin, aminoglycoside, nitrofurantoin, penicillins such as dicloxacillin, oxacillin, and sulfonamides; metronidazole, 5-fluoruracil, cisplatin, vinblastine, vincristine, epothilones, endostatin, verapamil, statins such as cerivastatin, atorvastatin, simvastatin, fluvastatin, rosuvastatin, pravastatin, and lovastatin, angiostatin; angiopeptin, taxanes such as paclitaxel; immunosuppressants or modulators such as rapamycin or derivatives thereof, such as biolimus, everolimus, ridaforolimus (previously known as deforolimus), Novolimus, Myolimus, temsirolimus, methotrexate, colchicine, flavopiridol, suramin, cyclosporin A, clotrimazole, flucytosine, griseofulvin, ketoconazole, miconazole, nystatin, terbinafine, steroids such as dexamethasone, prednisolone, corticosterone, budesonide, estrogen, hydrocortisone, and mesalamine; sulfasalazine, heparin and derivatives thereof, urokinase, PPack, argatroban, aspirin, abciximab, synthetic antithrombin, bivalirudin, enoxoparin, hirudin, r-hirudin, protamine, prourokinase, streptokinase, warfarin, flavonoids such as 7,3′,4′-trimethoxyflavone, and dipyramidole, trapidil, and nitroprusside. Other compounds may also be used.

The further pharmaceutical active substance or substances may be used individually or in combination in the same or different concentrations.

The further pharmaceutical active substance may be contained in a pharmaceutically active concentration of 0.2 to 3.5 μg/mm² implant surface, further 0.5 to 2.4 μg/mm² implant surface, or in other concentrations which may be less than 0.2 μg/mm² or greater than 3.5 μg/mm²

A combination of the at least one nitrostatin with one or more antiproliferative active substances is believed to be of particularly utility in at least some applications. One particular embodiment of promise is the combination of the at least one nitrostatin with at least one of the further pharmaceutical active substances paclitaxel and rapamycin, individually or in combination.

A combination to be used may include at least one nitrostatin with one or more active substances which influence the mTOR pathway. A combination may further include at least one nitrostatin with at least one of the further pharmaceutical active substances: sirolimus, everolimus, biolimus A9, Myolimus, Novolimus, zotarolimus, or other related active substances, as well as nucleic acid-based active substances which via RNA interference, for example, individually or in combination influence the mTOR pathway. Influencing the mTOR pathway allows the proliferative reaction of the vessel wall to be efficiently modified to the implant. One further particular advantage of a combination at least one mTOR-influencing active substance with at least one nitrostatin lies in the synergistic antiproliferative and anti-inflammatory effect, supplemented by antithrombotic and vasodilative effects.

A combination to be used may also include at least one nitrostatin with one or more active substances which interact with microtubules or their components. A combination may further include at least one nitrostatin with at least one of the further pharmaceutical active substances paclitaxel, docetaxel, and other taxanes and related active substances; active substances from the group of epothilones; and nucleic acid-based active substances which via RNA interference, for example, individually or in combination influence the synthesis, development, structure, or degradation of the microtubules. Influencing the microtubules allows the proliferative reaction of the vessel wall to be efficiently modified to the implant. The additional particular advantage of a combination of at least one microtubule-influencing active substance with at least one nitrostatin lies in the synergistic antiproliferative effect, supplemented by anti-inflammatory, antithrombotic, and vasodilative effects.

In one embodiment, the at least one nitrostatin and the further pharmaceutical active substance have different release kinetics than the coating of the implant in the surrounding implant tissue. For example, the various active substances may be present in different layers in the coating, the optionally used carrier matrix of the particular active substance being selected in such a way that the further pharmaceutical active substance is released from the carrier matrix more rapidly than the nitrostatin, or vice versa.

In a further embodiment, the implant according to the invention is a stent, and the further pharmaceutical active substance, which may be for example active substances from the class of antiproliferative agents, are delivered to the implant tissue using a coated balloon catheter. In addition, the further pharmaceutical active substance may be embedded in a biodegradable carrier matrix, for example a hydrogel or a polysaccharide, whereby during expansion of the balloon the carrier matrix is pressed against the tissue and remains adherent to the tissue after the balloon is deflated. It is thus ensured that the further pharmaceutical active substance is controllably released in a specified concentration.

In a further aspect some embodiments of the invention relate to the use of at least one nitrostatin in a coating of a medical implant, the coating completely or partially covering the surface of the implant.

The use of at least one nitrostatin in a coating of a medical implant has the dual advantage that, on the one hand, the surrounding implant tissue which is in contact with the coating of the implant experiences a superior antithrombotic, anti-inflammatory, and antiproliferative effect as the result of the statin component of this active substance, and on the other hand, the additional release of the NO component from this active substance counteracts vasoconstriction directly at the site because of the release of active substance from the coating of the implant according to the invention. These and other advantages provide unique benefits and advantages not achieved in the prior art.

In particular, when a medical implant of the prior art is made of a biocorrodible active substance such as magnesium or an alloy thereof, localized vasospasms may occur due to alkalosis caused by the degradation products of the active substance. This may result in undesirable shortening of the service life of the implant. By use of at least one nitrostatin in the implants according to the invention, it is possible to manufacture implants which are less sensitive to the effects of alkalosis on the implant. Longer service lives of the implants may thus be achieved. If the implant according to the invention is a stent, the risk of restenosis or damage of the vessel lumen in and around the stent region may be greatly reduced. Thus, by use of the implant the therapeutic aim being pursued may be achieved more satisfactorily, over a longer period of time, and with fewer side effects.

In a further aspect the invention relates to nitrostatins for the prophylaxis or therapy of restenosis or damage of the vessel lumen in a vessel section which has been provided with a stent, and the use of nitrostatins for such prophylaxis or therapy.

The invention further relates to nitrostatins for the prophylaxis or therapy of localized vasospasms, and the use of nitrostatins for such prophylaxis or therapy.

The invention is explained in greater detail below with reference to example embodiments which are not intended to limit the subject matter of the invention.

### Example Embodiment 1

### Coating a Stent with a Nitrostatin Contained in a Carrier Matrix

PLGA was used as carrier matrix. In the first step a 4% solution of PLGA in ethyl acetate was prepared (in a ratio of 65% L-lactide and 35% glycolide; the inherent viscosity of this polymer was 0.5-0.8 dL/g).

0.53 mg NCX 6560 was solubilized in 11.1 mL in the ethyl acetate polymer solution prepared above. NCX 6560 is a nitric oxide-releasing derivative of atorvastatin developed by NicOx S. A., Les Taissounières, Bât HB4,1681 route des Dolines, BP313, 06906 Sophia Antipolis cedex, France. The resulting solution was applied to the stent using a dipping process. The surface loading of NCX6560 was 0.75 μg/mm².

### Example Embodiment 2

### Cavity Filling of a Stent with a Nitrostatin Contained in a Carrier Matrix

The PLGA solution prepared in example embodiment 1 was used as carrier matrix.

0.60 mg active substance NCX6560 was solubilized in the PLGA:ethyl acetate polymer solution. The resulting solution was introduced into the cavities of the stent by microinjection. In addition, a coating of at least one nitrostatin may then be applied to the stent using a dipping process. The surface loading of NCX6560 was 0.5-1.6 μg/mm².

Example stents as described in Examples 1 and 2, as well as methods for manufacturing stents according to these example methods, lead to significant advantages and benefits as generally discussed above.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A medical implant, having a surface that is at least partially covered by a coating comprising at least one active substance, that comprises a nitrostatin.

2. A medical implant according to claim 1, characterized in that the nitrostatin is a compound of general formula (I) or a pharmaceutically acceptable salt or stereoisomer thereof 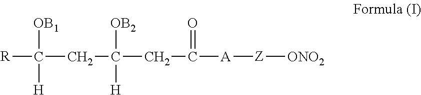
Wherein:
R is 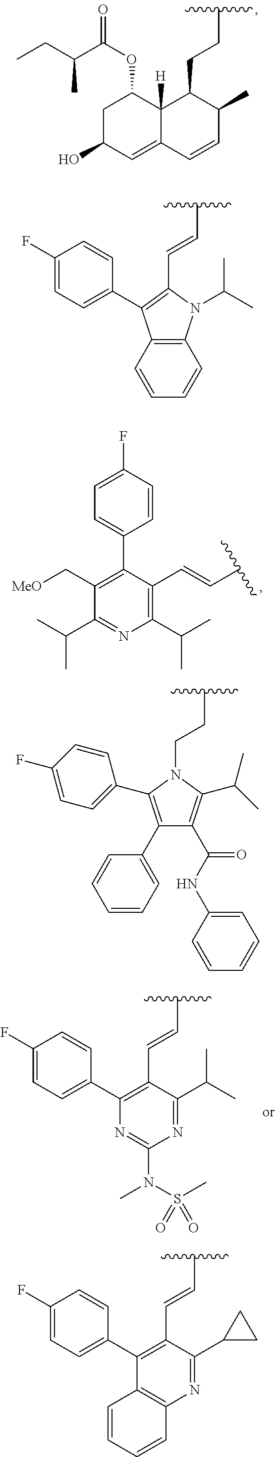
B₁ and B₂ are:
a) a further NO-releasing group comprising one or more of —NO₂, —ONO₂, or cinidomine (II),
b) a tosylate or mesylate group,
c) a straight-chain or branched C₁ to C₂₀ alkyl, optionally substituted with one or more substituents selected from the group comprising the following: halogen atoms, hydroxy, —ONO,
d) a heterocyclic saturated, unsaturated, or aromatic ring containing at least one heteroatom selected from nitrogen, oxygen, sulfur, and halogen atoms; the ring being additionally substituted with side chains having a straight-chain or branched alkyl containing 1 to 10 carbon atoms,
e) a cycloalkylene or cycloarylene ring containing 5 to 7 carbon atoms, the ring being substituted with side chains having a straight-chain or branched alkyl containing 1 to 10 carbon atoms, and optionally containing at least one heteroatoms selected from nitrogen, oxygen, sulfur, and halogen atoms,
wherein B₁ is the same as or different from B₂;
A is a straight-chain or branched C₁ to C₂₀ alkylene, —O—, —S—, —NH—, or NR¹, wherein
R¹ is a straight-chain or branched C₁-C₁₀ alkyl;
Z is a bivalent radical having the following meaning:
a) a straight-chain or branched C₁-C₂₀ alkylene, optionally substituted with at least one substituents selected from the following group: halide, —OH, —OR¹, —COOH, —COOR¹, —NH₂, NHR¹, NR¹R², wherein R¹ and R² are the same or different, and are a straight-chain or branched C₁-C₁₀ alkyl,
b) a cycloalkylene ring containing 5 to 7 carbon atoms, the ring optionally substituted with one or more straight-chain or branched C₁-C₁₀ alkyl side chains,
c) an arylalkylene having the formula:
wherein R³ is (CH₂)ₙ₁, where n1=0 or 20; R⁴ is —COOH, —COOR¹, —OH, —OR¹, where R¹ is defined as above; X is a saturated or unsaturated —(CH₂)ₘ group, where m=0-8; Y═O—CO—, —COO—; and R⁵ is (CH₂)ₙ₂, where n2=1-20,
d) a heterocycloalkylene having a saturated, unsaturated, or aromatic 5- or 6-membered ring containing one or more heteroatoms selected from nitrogen, oxygen, or sulfur, selected from the following groups:
e) —O—, —Sᵢ—, —Se—, —NH—, or NR¹, where i is an integer between 1 and 8 and R¹ is a straight-chain or branched C₁-C₁₀ alkyl, preferably CH₃.

3. A medical implant according to claim 1, characterized in that the implant is a stent.

4. A medical implant according to claim 3, characterized in that the stent is made of a biocorrodible metallic material.

5. A medical implant according to claim 4, characterized in that the stent is made of a magnesium alloy.

6. A medical implant according to claim 1, characterized in that the at least one nitrostatin is embedded in a polymeric carrier matrix.

7. A medical implant according to claim 1, characterized in that the at least one nitrostatin is present in a concentration of 0.2 to 3.5 μg/mm² implant surface.

8. A medical implant according to claim 1, characterized in that the coating contains at least one further pharmaceutically active substances selected from the group comprising: antimicrobial, antimitotic, antimyotic, antineoplastic, antiphlogistic, antiproliferative, antithrombotic, and vasodilatory active substances.

9. An implant according to claim 1 made through the steps of coating at least a portion of the implant with an active substance comprising a nitrostatin.

10. Nitrostatins for the prophylaxis or therapy of localized vasospasms.

11. A stent having a coating comprising nitrostatins for the prophylaxis or therapy of restenosis or damage of the vessel lumen in a vessel section.

12. A medical implant according to claim 7 wherein the concentration is between 0.5 and 1.6 μg/mm² implant surface.

13. A medical implant according to claim 8, wherein one or more of the further pharmaceutically active substances influences the mTOR pathway.

14. A medical implant according to claim 8, wherein one or more of the further pharmaceutically active substances interacts with microtubules.

15. A medical implant according to claim 8, wherein the one or more nitrostatin and the one or more further pharmaceutically active substances have release kinetics values such that at least one release kinetics value is different from at least one other release kinetics value.

16. A medical implant according to claim 8 wherein:
the at least one nitrostatins are provided in an amount of between about 0.2 to 3.5 μg/mm² implant surface; and,
the pharmaceutical active substance is provided in a concentration of 0.2 to 3.5 μg/mm² implant surface.

17. A medical implant according to claim 1, wherein the nitrostatin is a compound of general formula (I) or a pharmaceutically acceptable salt or stereoisomer thereof 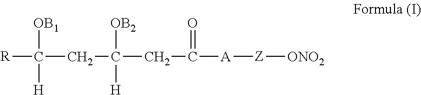
wherein:
R is 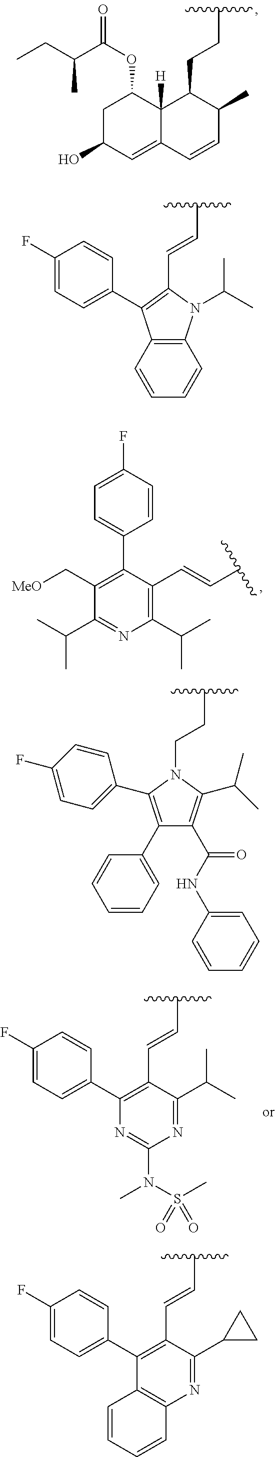
B₁ and B₂ are:
a) a further NO-releasing group,
b) a tosylate or mesylate group,
c) a straight-chain or branched C₁ to C₂₀ alkyl,
d) a heterocyclic ring; and
e) a cycloalkylene or cycloarylene ring.
A is a straight-chain or branched C₁ to C₂₀ alkylene;
Z is a bivalent radical selected from the group comprising:
a) a straight-chain or branched C₁-C₂₀ alkylene,
b) a cycloalkylene ring containing 5 to 7 carbon atoms,
c) an arylalkylene,
d) a heterocycloalkylene having a saturated, unsaturated, or aromatic 5- or 6-membered ring containing one or more heteroatoms selected from nitrogen, oxygen, or sulfur,
e) —O—, —Sᵢ—, —Se—, —NH—, or NR¹, where i is an integer between 1 and 8 and R¹ is a straight-chain or branched C₁-C₁₀ alkyl.

18. A medical implant according to claim 1, wherein:
the implant has a base body made of a magnesium alloy including at least 70% (wt.) magnesium and also including 3.7-5.5% (wt) yttrium;
the coating is provided in a thickness of between 1 nm to 100 μm; and,
the coating further comprises a carrier matrix that is configured to release the nitrostatin after the implant has been positioned in a desired implant location over a period of greater than 6 weeks.

19. A medical implant according to claim 1, wherein
the implant has a base body made of a magnesium alloy including at least 50% (wt.) magnesium and also including at least one rare earth metal;
the nitrostatin is present in a weight percentage of between 10% to 25% in the coating;
the coating further comprises at least one pharmaceutically active substance selected from the group comprising: antimicrobial, antimitotic, antimyotic, antineoplastic, antiphlogistic, antiproliferative, antithrombotic, and vasodilatory active substances; and,
the coating has a thickness of between about 5 μm to 60 μm.

20. A medical implant according to claim 1, wherein
the implant has a base body made of a magnesium alloy including at least one rare earth metal;
the nitrostatin is present in a weight percentage of less than 30% in the coating;
the coating has a thickness of between about 300 nm to 15 μm; and,
the coating further comprises a polymer matrix that forms a carrier matrix, the polymer matrix provided in a weight percentage of at least 70% and configured to release the nitrostatin over n extended period of time when implanted in a biologic environment.
